(19) United States
(12) Patent Application Publication
(10) Pub. No.: US 2025/0345578 A1
(43) Pub. Date: Nov. 13, 2025

(54) PHYSIOLOGICAL ACTION PROMOTION METHOD AND GOGGLES FOR HYDROGEN SUPPLY

(71) Applicant: Aqua Bank CO.,LTD., Osaka-shi, Osaka (JP)

(72) Inventor: Takashi TAKEHARA, Osaka-shi, Osaka (JP)

(21) Appl. No.: 18/855,711

(22) PCT Filed: Apr. 18, 2023
(86) PCT No.: PCT/JP2023/015450

§ 371 Date: Oct. 10, 2024

(30) Foreign Application Priority Data
Apr. 28, 2022 (JP) 2022-074866

(51) Int. Cl. A61M 35/00

(52) U.S. Cl. CPC A61M 35/30; A61M 2202/02; A61M 2210/0612

(57) ABSTRACT

Provided is a method for promoting orbicularis oculi blood flow by causing percutaneous absorption of hydrogen from a periphery of an eye, a method for promoting other physiological effects, and an optimal configuration of goggles for supplying hydrogen carrying out these methods. Further, the goggles provided hereunder include a goggle body for integrally covering and sealing a periphery of both eyes including at least a lower eyelid part of a user, a temple member coupled with both side parts of the goggle body, to be hooked on both ears of the user, and a hydrogen releasing part arranged at positions near both lower eyelid parts of the user inside the goggle body, having a pair of hydrogen releasing openings directed to each of the both lower eyelid parts, which are fluidly coupled with a discharging side end part of a tubular tube member connected to an external hydrogen generating apparatus.

### TECHNICAL FIELD

The present invention relates to a method for promoting physiological action, the method releasing air containing high-concentration hydrogen to a periphery of an eye, particularly a lower eyelid part, and causing percutaneous absorption to promote various kinds of physiological action, and to goggles for supplying hydrogen used for practical use of the method for promoting such physiological action.

### BACKGROUND ART

In recent years, effectiveness of hydrogen has been attracting attention, and various researches have been actively conducted. Hydrogen is regarded as having utility in removing only bad active oxygen (=hydroxyl radical), which is the cause of various diseases, etc., from the body, and since hydrogen does not affect the tissues or cells of the body, there is a wide range of methods to take hydrogen inside the body such as intravenous dosage, oral administration of an aqueous solution, and gas inhalation. In particular, the Applicant and the inventor have provided various results of clinical trials focusing on the effect of hydrogen intake by suction on the body and mind, and have also regularly provided a hydrogen suction apparatus allowing intake of gas containing high-concentration hydrogen (see Patent Literature 1).

On the other hand, conventionally, methods for taking hydrogen into a human body are mainly intake by drinking water containing high-concentration hydrogen and intake by suction of gas containing high-concentration hydrogen, which take hydrogen into blood through the gastrointestine and the lung. In such situation, the Applicant and the inventor have understood that suction intake of gas containing high-concentration hydrogen dramatically promotes nerve activities/blood circulation activities of living organisms (see Patent Literature 2), and have still continued developing effects of hydrogen suction on living organisms and an appropriate hydrogen suction apparatus. In the process of development, the inventor has also realized that quick delivery of blood containing high-concentration hydrogen to each region is important in hydrogen intake, and has been examining/demonstrating methods for causing direct delivery to each region. Among such methods, an effect on eyesight caused by hydrogen intake is one of effects that can be seen in a short time, and thus since direct release of hydrogen to an eye for intake is considered to be effective, goggles for supplying hydrogen to a periphery of an eyeball are provided (International Application PCT/JP2021/040625).

These goggles refer to a conventionally existed clinical trial that an effect of hydrogen to an eyeball includes an effect on retina oxidative stress damage, etc., resulted from instillation of water containing hydrogen, and based on a hypothesis that direct release of hydrogen to an originally moisturized eyeball to let high-concentration hydrogen water be a moisture of the surface of the eyeball is more effective, dedicated goggles allowing supply of high-concentration hydrogen are developed, and beneficial effects are actually obtained.

However, subsequent demonstrations demonstrated mechanisms, optimal intake regions, physiological effects caused by hydrogen intake regarding hydrogen intake from around an eyeball, and it has been realized that supplying hydrogen directly to an eyeball is not the optimal way.

### CITATION LIST

### Patent Literature

Patent Literature 1: International Publication No. WO 2018/047889
Patent Literature 2: International Publication No. WO 2018/151107

### SUMMARY OF INVENTION

### Technical Problem

The present invention has been made in view of such circumstance, and its purpose is to provide a method for promoting orbicularis oculi blood flow by causing percutaneous absorption of hydrogen from a periphery of an eye, a method for promoting other physiological effects, and an optimal configuration of goggles for supplying hydrogen to carry out such methods.

### Solution to Problem

A first present invention created to solve the above-described problem provides a method for promoting physiological action, the method releasing air containing high-concentration hydrogen to a periphery of an eye, and causing percutaneous absorption of hydrogen to promote orbicularis oculi blood flow.

The first present invention is provided from a knowledge that, when hydrogen is supplied to a periphery of an eye, percutaneous absorption of its hydrogen component from the periphery of the eye improves various kinds of physiological action. As described above, conventionally, a physiological effect caused by supply of hydrogen to a periphery of an eye has been based on a hypothesis that supply of hydrogen to an eyeball in a moisturized state achieves improved effectiveness by extension of an effect of instilling a solution containing hydrogen. However, in a clinical trial upon developing the present invention, supply of high-concentration hydrogen to a periphery of an eye has a large influence on orbicularis oculi blood flow, and it is found out that “percutaneous absorption” from skin in the periphery of the eye promotes the “orbicularis oculi blood flow”, resulting in exertion of various physiological effects. Based on this knowledge, the present invention provides, as an effective method for supplying hydrogen to an eye and a periphery of the eye, a method for promoting orbicularis oculi blood flow by supplying high-concentration hydrogen to a periphery of an eye, and causing absorption (percutaneous absorption) from the skin in the periphery of the eye.

In particular, as a place in the periphery of the eye to be subjected to percutaneous absorption of the air containing high-concentration hydrogen, the first present invention representatively provides a method for promoting physiological action, the method releasing air containing high-concentration hydrogen to a lower eyelid part, and causing percutaneous absorption of hydrogen to promote orbicularis oculi blood flow.

The present method for promoting physiological action is provided as a preferable example of the present invention based on a knowledge that, among the periphery of the eye, the lower eyelid part has a larger effect in promoting orbicularis oculi blood flow when being subjected to percutaneous absorption of hydrogen.

In addition, as an example of specific another kind of physiological action on which the aforementioned present invention, which causes percutaneous absorption of the high-concentration hydrogen from the periphery of the eye, exerts a significant effect, a method for promoting physiological action, the method releasing air containing high-concentration hydrogen to a periphery of an eye, and causing percutaneous absorption of hydrogen to reduce brain fatigue, is provided.

In addition, as another kind of physiological action on which the method for promoting physiological action of the present invention exerts a significant effect, a method for promoting physiological action, the method releasing air containing high-concentration hydrogen to a periphery of an eye, and causing percutaneous absorption of hydrogen to enhance brain functions (improve age), is provided.

Moreover, as further another kind of physiological action on which the method for promoting physiological action of the present invention exerts a significant effect, a method for promoting physiological action, the method releasing air containing high-concentration hydrogen to a periphery of an eye, and causing percutaneous absorption of hydrogen to promote oxytocin secretion, is provided.

Note that, as in the case of promotion of orbicularis oculi blood flow, the hypothesis that releasing air containing high-concentration hydrogen to a lower eyelid part and causing percutaneous absorption is preferable can be obtained also when utilizing the present method for promoting physiological action in the aforementioned reduction in brain fatigue, enhancement of brain functions (age improvement), and promotion of oxytocin secretion.

Next, a second present invention provides goggles for supplying hydrogen having an optimal configuration for the first present invention as the aforementioned method for promoting physiological action, which promotes orbicularis oculi blood flow by causing percutaneous absorption of the high-concentration hydrogen from the periphery of the eye (in particular, the lower eyelid part). Specifically, the second present invention provides goggles for supplying hydrogen, including:
a goggle body (for example, see a goggle body 12 in the embodiment) for integrally covering and sealing a periphery of both eyes including at least a lower eyelid part of a user;
a temple member (for example, see a temple member 15 in the embodiment) coupled with both side parts of the goggle body, to be hooked on both ears of the user; and
a hydrogen releasing part (for example, see a bottom hydrogen releasing part 124 in the embodiment) arranged at positions near both lower eyelid parts of the user inside the goggle body, having a pair of hydrogen releasing openings (for example, see a hydrogen releasing opening 124a in the embodiment) directed to each of the both lower eyelid parts, which are fluidly coupled with a discharging side end part of a tubular tube member (for example, see a tube member 16 in the embodiment) connected to an external hydrogen generating apparatus.

Since it has been discovered that percutaneous absorption of high-concentration hydrogen from a periphery of an eye as provided in the first present invention is effective in promotion of orbicularis oculi blood flow, reduction in brain fatigue, enhancement of brain functions (age improvement), and promotion of oxytocin secretion, the second present invention provides a goggle-type hydrogen supply apparatus, which becomes airtight at the time of wearing, as an optimal apparatus configuration for causing percutaneous absorption of the high-concentration hydrogen from the periphery of the eye such that the hydrogen of a high-concentration state sufficiently spreads over the skin in the periphery of the eye. In particular, there is also a characteristic in that a pair of hydrogen releasing parts directed to each of both lower eyelid parts are provided such that hydrogen can be directly released to both lower eyelid parts, which are discovered to be regions receiving an especially large effect from percutaneous absorption of hydrogen.

In addition, the above-described goggles for supplying hydrogen preferably has a configuration in which
a coupling member (for example, see a coupling member 14 in the embodiment) for allowing inside and outside communication is arranged in both side parts of the goggle body, the goggle body and the temple member being coupled by coupling of a tip of each temple member with the coupling member,
the coupling member includes, in order from an inner side,
   an inner coupling part (for example, see a temple inner cap 141 in the embodiment) on the inner side of the goggle body,
   a pedestal part (for example, see a pedestal part 142 in the embodiment) to be mounted on both sides of the goggle body,
   an outer coupling part (for example, see a temple coupling part 143 in the embodiment) which is coupled with the goggle body by sandwiching the pedestal part with the inner coupling part from both sides for coupling, the outer coupling part also being coupled with the temple member, and
   an outer coupling part (for example, see a temple outer cap 144 in the embodiment) coupled with the outer coupling part, which is fluidly connected with the discharging side end part of the tube member,
the inner coupling part, the pedestal part, the outer coupling part, and the outer coupling part fluidly connect the discharging side end part of the tube member and an inside of the goggles while being coupled to one another (for example, see communication from a recessed part 144c to a releasing part 141b in the embodiment), and
a main body part (for example, a main body part 151 in the embodiment) of the temple member has a detaining part (for example, a detaining part 152 in the embodiment) for detachably positioning the tube member.

In the preferable present goggles for supplying hydrogen, instead of directly coupling the goggle body with the temple member, the goggle body and the temple member have an assembly structure in which the coupling member as a member for coupling is mechanistically interposed. The coupling member, which can be used in common as an assembly member, also has a function of supplying high-concentration hydrogen from the tube member into the goggle body of an airtight state to achieve a fluid connection. This coupling member can put the structural coupling function and the fluidly coupling function together in one member, and it allows provision of goggles for supplying hydrogen realizing a simple assembly, while sufficient filling of high-concentration hydrogen is facilitated. As a result, a user having a portable hydrogen supply apparatus can percutaneously absorb high-concentration hydrogen from a periphery of an eye of the user with simple and inexpensive goggles. Moreover, since the detaining part of the tube member is provided for the temple member coupled with this coupling member, the tube member can be securely positioned with the detaining part only by inserting the discharging side end part of the tube member into the coupling member, and hydrogen can be guided into the goggle body from either the right side or the left side depending on the selection of the user, thereby allowing provision of a situation in which it is easy to habitually use hydrogen in accordance with the posture and the condition of the user, whether the dominant hand of the user being the right or the left, a portable hydrogen supplier, etc. In addition, by applying such configuration and preparing a plurality of types of temple members and goggle bodies, while using a common coupling member, it is possible to provide an easy custom-made product with a reduced manufacturing cost by preparing and combining a minimum component lineup in accordance with the head size and the position of the eye of the user.

Moreover, it is preferable that
the goggle body includes an edge part (for example, an edge part 122 in the embodiment) which contacts a periphery of an eye of the user, and seals an inside and an outside of the goggle body, while providing a space between a front lens part (for example, a lens part 123 in the embodiment) and the periphery of the eye,
the hydrogen releasing part, which is adjustable to a position corresponding to each of the both lower eyelid parts, is arranged on a goggle body lower part side of the edge part, and
each hydrogen releasing part is fluidly connectable using a detachable short tube member (for example, a short tube member 16 in the embodiment), and either of the hydrogen releasing openings is fluidly connectable with a through hole, to which high-concentration hydrogen is supplied, using another detachable short tube member.

According to the embodiment of the goggles for supplying hydrogen, based on a thought to directly release high-concentration hydrogen to a lower eyelid part, which is especially effective for promotion of orbicularis oculi blood flow, etc., and causing percutaneous absorption, the hydrogen releasing part is made adjustable in accordance with individual users. Moreover, other regions in the periphery of the eye inside the goggle body are subjected to percutaneous absorption with hydrogen spread inside the goggle body through the lower eyelid part. In addition, this configuration allows the most efficient hydrogen intake in accordance with the face and the head size/shape of the user.

### Advantageous Effects of Invention

The method for promoting physiological effects of the first present invention can achieve a method for promoting orbicularis oculi blood flow by causing percutaneous absorption of hydrogen from a periphery of an eye, and promotion of other physiological effects. In addition, the goggles for supplying hydrogen of the second present invention can provide goggles for supplying hydrogen optimal for carrying out the method for promoting physiological effects of the first present invention, which is simple and has a small number of components, while meeting various types of user's requirements.

### BRIEF DESCRIPTION OF DRAWINGS

FIGS. 1A-1B show results of analyzing an influence of percutaneous absorption of high-concentration hydrogen from each region in a periphery of an eye, on orbicularis oculi blood flow. FIG. 1A shows positions of each region A to E to be subjected to percutaneous absorption of the high-concentration hydrogen, and FIG. 1B shows an average value of amounts of change in the blood flow in both eyes at positions A to E.

FIGS. 2A-2B show results of analyzing an influence of percutaneous absorption of the high-concentration hydrogen from the periphery of the eye, on degrees of brain fatigue. FIG. 2A shows the state of a flicker measurement adopted as measuring of a degree of brain fatigue, and FIG. 2B shows a graph chart comparing flicker values (frequency Hz) when causing percutaneous absorption of environmental air from the periphery of the eye (control (air)) and when causing percutaneous absorption of the high-concentration hydrogen from the periphery of the eye (hydrogen).

FIGS. 3A-3B show results of analyzing an influence of percutaneous absorption of the high-concentration hydrogen from the periphery of the eye, on brain age.

FIGS. 4A-4C show results of analyzing an influence of percutaneous absorption of the high-concentration hydrogen from the periphery of the eye, on brain waves.

FIG. 5 shows, as results of analyzing an influence of percutaneous absorption of the high-concentration hydrogen from the periphery of the eye, on salivary oxytocin secretion, a graph chart comparing amounts of change in oxytocin when causing percutaneous absorption of environmental air from the periphery of the eye (control (air)) and when causing percutaneous absorption of the high-concentration hydrogen from the periphery of the eye for eight minutes (hydrogen).

FIG. 6 is a perspective view (photographic diagram) of an embodiment of goggles for supplying hydrogen according to a second present invention, seen from the upper right front.

FIG. 7 is an assembly and disassembly perspective view (photographic diagram) of each member in the present embodiment of the goggles for supplying hydrogen, seen from the upper right front.

FIG. 8 is a perspective view (photographic diagram) of a goggle body and a temple member in the present embodiment of the goggles for supplying hydrogen, seen from the upper right back.

### DESCRIPTION OF EMBODIMENTS

Firstly, an example of a method for promoting physiological action which promotes orbicularis oculi blood flow (hereinafter, also simply referred to as the “present method for promoting physiological action”) according to a first present invention, and its demonstration, will be described below.

Regarding Verification Results of Physiological Action Due to Percutaneous Absorption of High-Concentration Hydrogen from a Periphery of an Eye, and the Present Method for Promoting Physiological Action Based on the Verification Results>>

In a verification test using the present method for promoting physiological action, as will be shown below, physiological effects due to percutaneous absorption of high-concentration hydrogen from a periphery of an eye were verified. Verification conditions in the verification test were as follows.

Test participant: healthy women in her twenties or thirties (living in or near Tokyo, n=24)
Sample to be examined: new-type hydrogen generating apparatus
Contents to be examined: Analyses of action on autonomic nerve activities, action on central nerve activities, changes in hemodynamics, action on dynamic visions, action on emotions/feelings, etc.
Usage: generating hydrogen of 50 cc/min from the new-type hydrogen generating apparatus, and guiding the generated hydrogen to dedicated eye goggles. Use for eight minutes (measure brain waves during use), then evaluate autonomic nerves, etc.
Items to be investigated: central nerve activities (measurement of brain waves), dynamic vision measurement, emotions/feelings evaluation (many-sided emotion level questionnaires), peripheral circulatory functions (a two-dimensional contactless blood flowmeter), evaluation of brain activities/brain fatigue (measurement of flicker values), evaluation of brain stress/brain age (ATMT measurement), and quantification of salivary oxytocin (ELISA method).

As a result of conducting the above-described verification test, although absorption of high-concentration hydrogen from an eyeball and a periphery of the eye has been conventionally based especially on a hypothesis that hydrogen permeation into moisture of the surface of the eyeball has a particularly significant influence on physiological action such as brain functions, etc., it became evident that percutaneous absorption of high-concentration hydrogen from the periphery of the eye has a large influence on orbicularis oculi blood flow. Firstly, this point will be described.

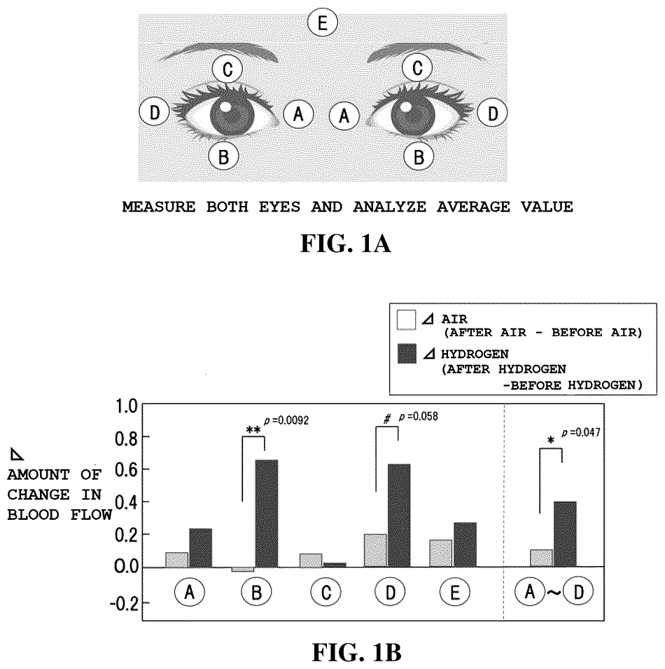

FIGS. 1A-1B show results of analyzing an influence of percutaneous absorption of high-concentration hydrogen from each region in a periphery of an eye, on orbicularis oculi blood flow. FIG. 1A shows positions of each region A to E to be subjected to percutaneous absorption of the high-concentration hydrogen, and FIG. 1B shows an average value of amounts of change in the blood flow in both eyes at positions A to E. The amounts of change in blood flow here were measured using OMEGAZONE (manufactured by OMEGAWAVE, INC.), which continuously displays a blood flow state of a body tissue by color distribution with a high definition and at a high speed. A represents inner corner parts of the eyes, B represents lower eyelid parts, C represents upper eyelid parts, D represents outer corner parts of the eyes, and E represents a forehead part (including a part between eyebrows).

The left side of bar graphs shown in parallel for each region A to E in FIG. 1B represents amounts of change in blood flow before and after percutaneous absorption of an environmental atmosphere (Δ air), and the right side of the bar graphs represents amount of change in blood flow before and after percutaneous absorption of the high-concentration hydrogen (Δ hydrogen). The bar graphs show A (inner corner parts of the eyes), B (lower eyelid parts), C (upper eyelid parts), D (outer corner parts of the eyes), E (forehead part), and an average value of A to D. In view of this graph chart, high concentration percutaneous absorption from the periphery of the eye is effective in promoting orbicularis oculi blood flow, and statistical significance can be seen especially in B (lower eyelid parts), D (outer corner parts of the eyes), and the average value of A to D. Among these, further remarkable significance is seen in B (lower eyelid parts), and the p value is also 0.0092, which is extremely small. From these points, the present invention provides releasing the air containing high-concentration hydrogen to the periphery of the eye, and causing percutaneous absorption of hydrogen, as a method for promoting physiological action which promotes orbicularis oculi blood flow, and as a particularly preferable method, direct release of high-concentration hydrogen to the lower eyelid parts is provided. Note that, P values displayed in FIG. 1B indicate a probability of a test statistic becoming that value on the basis of a null hypothesis in statistical hypothesis testing, and a smaller P value means that the test statistic is less likely to become that value.

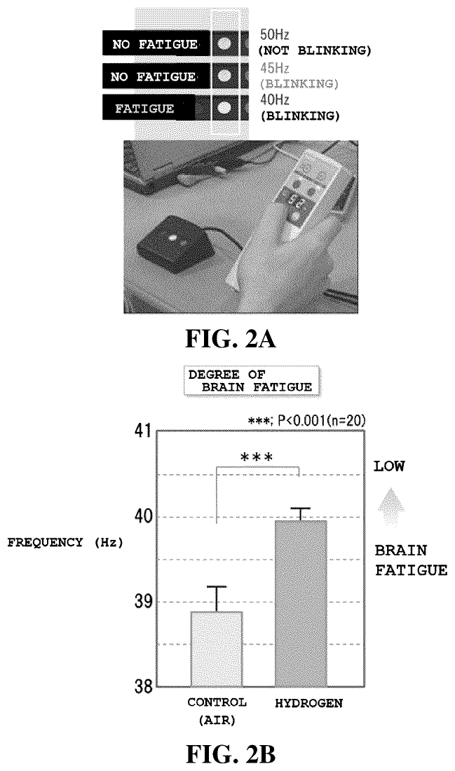

FIGS. 2A-2B shows results of analyzing an influence of percutaneous absorption of the high-concentration hydrogen from the periphery of the eye, on degrees of brain fatigue. FIG. 2A shows the state of a flicker measurement adopted as measuring of a degree of brain fatigue, and FIG. 2B shows a graph chart comparing flicker values (frequency Hz) when causing percutaneous absorption of environmental air from the periphery of the eye (control (air)) and when causing percutaneous absorption of the high-concentration hydrogen from the periphery of the eye (hydrogen). In this regard, a flicker value is a frequency that causes a blinking light to be in a borderline of being continuously seen or flickering, which is regarded as being closely related to an active state of the cerebral cortex carrying out advanced information processing, and this is particularly known as a method for measuring a degree of brain fatigue.

From the graph chart of FIG. 2B, after supply of hydrogen, a significant raise of the flicker value is observed as compared to the control, while the p value is also 0.001, which is extremely small, and it is suggested that percutaneous absorption of the high-concentration hydrogen from the periphery of the eye results in reduction of brain fatigue.

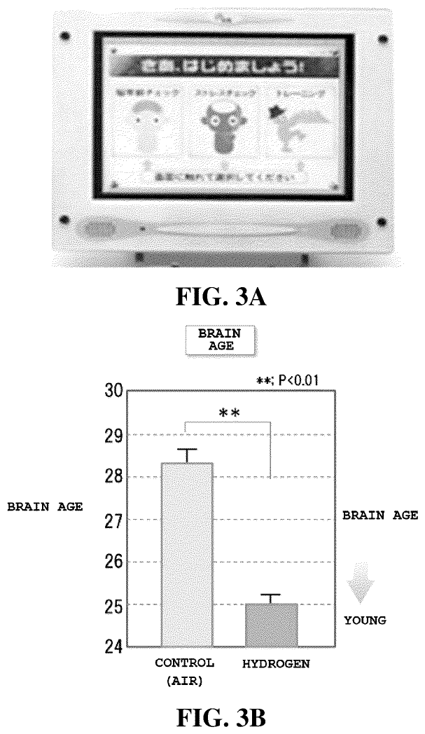

Next, FIGS. 3A-3B show results of analyzing an influence of percutaneous absorption of the high-concentration hydrogen from the periphery of the eye, on brain age. Brain age here was measured using Advanced Trail Making Test (ATMT: see FIG. 3A), which is developed in Laboratory of psychophysiology, Department of Psychiatry, Osaka University School of Medicine for the purpose of checking weakening of brain age. This ATMT is quantification of brain age (vitality, readiness, and effectiveness of a brain), and is one of representative means of evaluating brain age, which utilizes a characteristic that degradation of information processing ability is likely to be reflected on narrowed attentiveness and working memory to check health/aging like a game.

FIG. 3B shows a graph chart comparing brain age when causing percutaneous absorption of environmental air from the periphery of the eye (control (air)) and when causing percutaneous absorption of the high-concentration hydrogen from the periphery of the eye for eight minutes (hydrogen). From this graph chart, after supply of hydrogen, significant lowering of brain age is observed as compared to the control air, and it is suggested that brain functions are enhanced (age is improved) due to percutaneous absorption of the high-concentration hydrogen from the periphery of the eye.

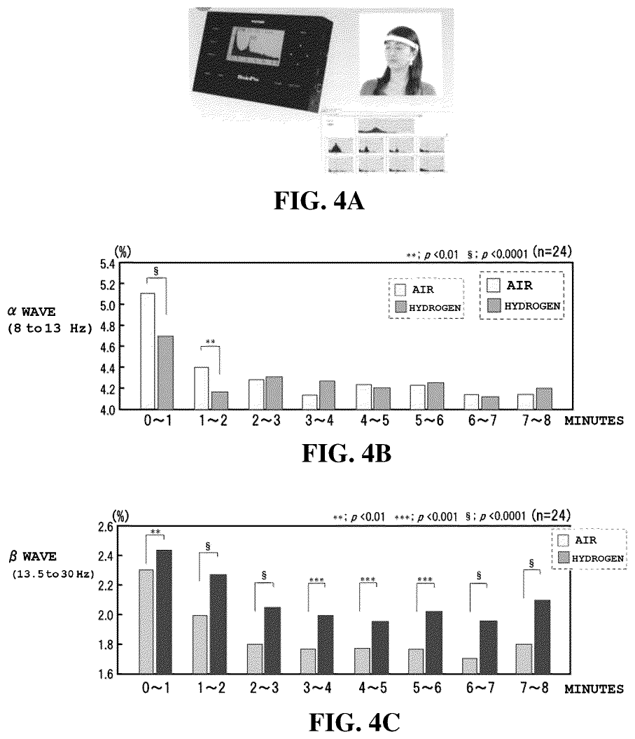

In addition, FIGS. 4A-4C show results of analyzing an influence of percutaneous absorption of the high-concentration hydrogen from the periphery of the eye, on brain waves. Brain waves here were measured using BrainPro (Futek Electronics Co., Ltd.) shown in FIG. 4A as a brain wave measuring system. The types of brain waves include α waves occurring around the back of the head (8 to 13 Hz) in a resting period/eye-closed state, β waves occurring near the front of the head/central gyrus (14 to 30 Hz) during mental activities, θ waves occurring during a light sleep (4 to 7 Hz), and high-amplitude slow waves δ waves occurring during a sleep (0.5 to 4 Hz). FIGS. 4B, 4C show graph charts displaying, in parallel, output ratios (%) of α waves and β waves for each percutaneous absorption time, comparing percutaneous absorption of environmental air from the periphery of the eye (control (air)) and percutaneous absorption of the high-concentration hydrogen from the periphery of the eye (hydrogen), respectively. From these graph charts, as compared to the air inflow, α waves are significantly decreased and β waves are significantly raised immediately after the hydrogen inflow, and thus activation of the cerebrum is suggested.

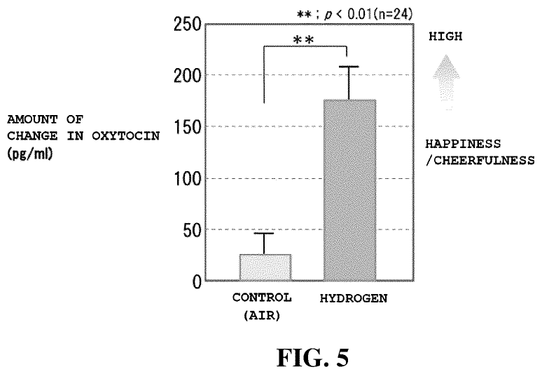

Moreover, FIG. 5 shows results of analyzing an influence of percutaneous absorption of the high-concentration hydrogen from the periphery of the eye, on salivary oxytocin secretion. In this regard, oxytocin is a hormone synthesized in neurosecretory cells of the nuclei paraventricularis and the supraoptic nuclei of the hypothalamus, which is secreted from the posterior pituitary. FIG. 5 shows a graph chart comparing amounts of change in oxytocin when causing percutaneous absorption of environmental air from the periphery of the eye (control (air)) and when causing percutaneous absorption of the high-concentration hydrogen from the periphery of the eye for eight minutes (hydrogen). From this graph chart, after supply of hydrogen, cheerful hormones (happy hormones) are significantly raised as compared to the control air, and it is suggested that positive emotions such as happiness, cheerfulness, and satisfaction are evoked due to percutaneous absorption of the high-concentration hydrogen from the periphery of the eye.

The analysis results as above suggest that percutaneous absorption of hydrogen from the periphery of the eye allows increase in blood flow in the periphery of the eye, maintenance/improvement of dynamic visions, decrease in a waves, increase in R waves, reduction in brain fatigue, activation of brain functions, and increase in salivary oxytocin. In view of these points, it is inferred that releasing the air containing high-concentration hydrogen to the lower eyelid parts for causing percutaneous absorption is preferable not only for promotion of orbicularis oculi blood flow according to the analysis results in FIGS. 1A-1B, but also for reduction in brain fatigue, enhancement of brain functions (age improvement), influence on brain waves, and promotion of oxytocin secretion.

### <<Regarding Goggles for Supplying Hydrogen According to the Present Invention>>

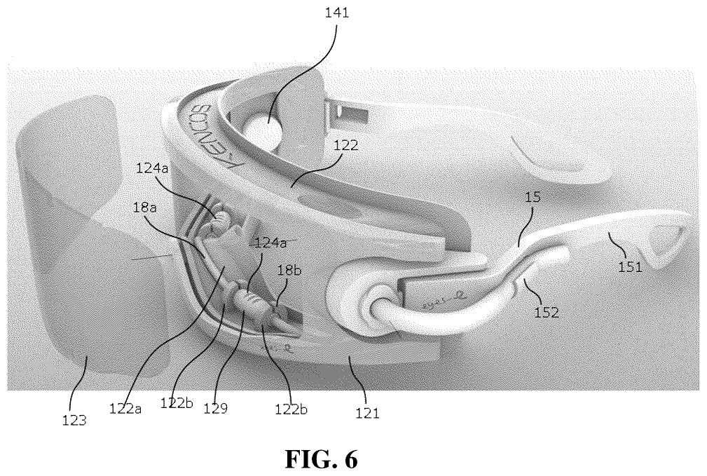

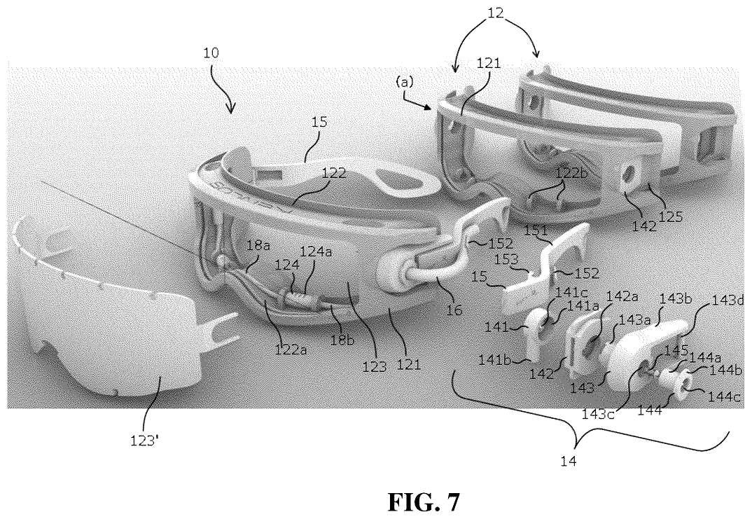

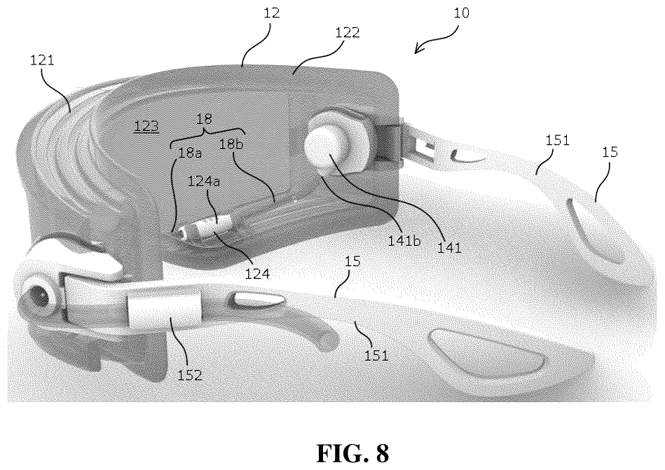

FIG. 6 is a perspective view (photographic diagram) of an embodiment 10 of the goggles for supplying hydrogen according to the second present invention, seen from the upper right front. FIG. 7 is an assembly and disassembly perspective view (photographic diagram) of each member of the embodiment 10 of the goggles for supplying hydrogen, seen from the upper right front. FIG. 8 is a perspective view (photographic diagram) of a goggle body and a temple member of the embodiment 10 of the goggles for supplying hydrogen, seen from the upper right back. Note that FIG. 7 also shows a lens protection member 123′, which covers the front face of a lens part 123 to protect the lens part 123 when storing/keeping the goggles for supplying hydrogen 10.

The goggles for supplying hydrogen 10 are constituted of a goggle body 12, a temple member 15, and a tube member 16. The goggle body 12 is a curved member projecting to the front and surrounding a periphery of an eye of a user to cover the front of the eye, and is an assembly body constituted of the lens part 123, which is a transparent body, a front frame 121 into which the lens part 123 is fitted, and an edge part 122 arranged along a periphery of the rear face of the front frame 121 such that the periphery of the rear face is fitted to (brought into contact with) the periphery of the eye. The lens part 123, the front frame 121, and the edge part 122 are assembled to seal the periphery of the eye of the user from the outside while ensuring the field of front vision, and to provide a space to allow filling of gas inside the goggle body 12. In particular, the edge part 122 is configured to cover a periphery of both eyes including the lower eyelid parts, and seal the skin of the periphery of both eyes within the goggle body 12.

In addition, a pair of substantially annular support parts 122b, which fix a periphery of both ends of a tubular member at a position corresponding to the lower side of each of both eyes of the user by snapping, are arranged in a lower edge part 122a on the lower side of the edge part 122, and a bottom hydrogen releasing part 124 coupled with a short tube member 18 is fixed to each of the pair of support parts 122b. The bottom hydrogen releasing part 124 is provided with a hydrogen releasing opening 124a for releasing hydrogen fed from the short tube member 18 into the goggle body 12. Moreover, one end of each bottom hydrogen releasing part 124 passes a bulged nose pad part in an intermediate part of the lower edge part 122a to be fluidly coupled with each other at a central short tube member 18a, and the another end is fluidly connected to a releasing part 141b of a temple inner cap (inner coupling part) 141 of a coupling member 14 to be described later. In this manner, hydrogen propagated from the external tube member 16 is released to the periphery of the eye of the user from each releasing opening 124a through the releasing part 141b, a lateral short tube member 18b, and the bottom hydrogen releasing part 124. Furthermore, since the pair of bottom hydrogen releasing parts 124 are fluidly connected to each other at the central short tube member 18a, regardless of the external tube member 16 being arranged (detained) in either the right or the left, hydrogen is released from the releasing openings 124a of both bottom hydrogen releasing parts 124. Accordingly, the user can detain the tube member 16 in either the right or the left of the goggles for supplying hydrogen 10, and since there is freedom in the dominant hand of the user and the portable position of a portable hydrogen supply apparatus to supply hydrogen to the goggles for supplying hydrogen 10, it is convenient.

Next, the coupling member 14 of the goggle body 12 and the temple member 15 will be described by referring mainly to FIG. 7. The coupling member 14 is generally constituted of the temple inner cap (inner coupling part) 141, a pedestal part 142, a temple coupling part 143, a temple outer cap (outer coupling part) 144, and a screw member 145, from the goggle body 12 side. The temple inner cap 141 covers, from the inner side, a small-diameter part 143a of the temple coupling part 143 penetrating the pedestal part 142 and projecting to the inner side, for coupling and fixing. The temple inner cap 141 is provided with the nozzle-shaped releasing part 141b connected to an end part of the lateral short tube member 18b (see FIG. 8), and its outer side is formed to have a counterbored part 141a, which receives the inner side of the pedestal part 142 and serves as a counterbore for coupling. This counterbored part 141a is a tubular recessed part opened to the outer side.

In addition, as shown in FIG. 7, the pedestal part 142 is fitted into a side recessed part 125 provided for both side parts of the front frame 121 so as to be positioned at both side parts of the goggle body 12, and is provided with a through hole 142a in a lateral direction. Moreover, the temple coupling part 143 is provided with a through hole (not shown) into which the screw member 145 can be inserted from the inner side, and is provided with a tubular recessed part 143c linked with the through hole and opened toward the outer side while increasing its diameter. With this configuration, the pedestal part 142 is sandwiched between the temple inner cap 141 and the temple coupling part 143 while being fitted into the side recessed part 125 of the front frame 121, and by fastening the screw member 145 into a screw hole 141c at the center of the counterbored part 141a of the temple inner cap 141, the temple inner cap 141, the pedestal part 142, and the temple coupling part 143 are coupled and fixed to the goggle body 12.

Furthermore, the temple outer cap (outer coupling part) 144 is constituted of a small-diameter part 144a and a large-diameter flange part 144b from the inner side, and is provided with a recessed part 144c opened to the outer side while being linked with a through hole (not shown) extending on the axis. The small-diameter part 144a of this temple outer cap 144 is inserted into the recessed part 143c of the temple coupling part 143, and while positioning is enabled in the inner side direction with the flange part 144b, one end of the tube member 16 is inserted and fixed into the recessed part 144c (fixing with elastic force/frictional force derive from resin), thereby causing hydrogen fed from a portable hydrogen supply apparatus (not shown) to be released and filled inside the goggle body 12 from the hydrogen releasing opening 124a of the bottom hydrogen releasing part 124 through the temple inner cap 141 and the short tube member 18.

Next, the temple member 15 to be hooked on an ear of the user, which is also referred to as a so-called temple of glasses, will be described. The temple member 15 is formed such that one end is coupled with the aforementioned coupling member 14, and the another end side has a hooking shape by being curved, thereby allowing the another end to be hooked on the ear while contacting the head of the user. In addition, the outer side of an intermediate part of a main body part 151 in the longitudinal direction is provided with a detaining part 152 for snapping the tube member 16, and by inserting and fixing the tip of the main body part 151 into the recessed part 144c of the temple outer cap 144, while supporting the tube member 16 at the position of the detaining part 152 separated therefrom, the tube member 16 can be securely fixed to the goggle body 12 and the coupling member 14.

Furthermore, the main body part 151 of the temple member 15 is a plate member having flexibility inward and outward such as a resin, and a hooking part 153 to be hooked from the front for allowing positioning to avoid displacement to the rear is arranged on the inner side of the intermediate position of the main body part 151 in the longitudinal direction. By hooking this hooking part 153 to a support part 153 arranged at the rear of the temple coupling part 143 from the front, positioning is enabled. As a result, the main body part 151 of the temple member 15 is warped outward and brought into contact with the head of the user due to restoration force inward, thereby allowing positioning in the inward-outward directions, while positioning in the front-rear directions is enabled using the support part 153 and the hooking part 153.

The method for promoting physiological action, etc., the method releasing air containing high-concentration hydrogen to a periphery of an eye, and causing percutaneous absorption of hydrogen to promote orbicularis oculi blood flow, and embodiments of the preferable goggles for supplying hydrogen according to the present invention have been exemplified and described above, but the present invention is not limited thereto, and it will be understood by those skilled in the art that other modifications and improvements can be made without departing from the spirit and the teaching described in the claims and the specification or the like.

### REFERENCE SIGNS LIST

10 goggles for supplying hydrogen
12 goggle body
14 coupling member
15 temple member
16 tube member
18 short tube member
18a central short tube member
18b lateral short tube member
121 front frame
122 edge part
122a lower edge part
122b support part
123 lens part
123′ lens protection member
124 bottom hydrogen releasing part
124a hydrogen releasing opening
125 side recessed part
128 plug member
141 inner coupling part (temple inner cap)
141a counterbored part
141b releasing part (not shown)
141c screw hole
142 pedestal part
142a through hole
143 temple coupling part
143a small-diameter part
143b main body part
143c recessed part
143d support part
144 outer coupling part (temple outer cap)
144a small-diameter part
144b flange part
144c recessed part
145 screw member
151 main body part
152 detaining part
153 hooking part

1. A method for promoting physiological action, the method releasing air containing high-concentration hydrogen to a periphery of an eye, and causing percutaneous absorption of hydrogen to promote orbicularis oculi blood flow.

2. The method for promoting physiological action according to claim 1, wherein the air containing high-concentration hydrogen is released to a lower eyelid part, and percutaneous absorption of the hydrogen is caused to promote the orbicularis oculi blood flow.

3. The method for promoting physiological action according to claim 1, wherein the air containing high-concentration hydrogen is released to the periphery of the eye, and percutaneous absorption of the hydrogen is caused to reduce brain fatigue.

4. The method for promoting physiological action according to claim 1, wherein the air containing high-concentration hydrogen is released to the periphery of the eye, and percutaneous absorption of the hydrogen is caused to enhance brain functions (improve age).

5. The method for promoting physiological action according to claim 1, wherein the air containing high-concentration hydrogen is released to the periphery of the eye, and percutaneous absorption of the hydrogen is caused to promote oxytocin secretion.

6. Goggles for supplying hydrogen, comprising:
a goggle body for integrally covering and sealing a periphery of both eyes including at least a lower eyelid part of a user;
a temple member coupled with both side parts of the goggle body, to be hooked on both ears of the user; and
a hydrogen releasing part arranged at positions near both lower eyelid parts of the user inside the goggle body, having a pair of hydrogen releasing openings directed to each of the both lower eyelid parts, which are fluidly coupled with a discharging side end part of a tubular tube member connected to an external hydrogen generating apparatus.

7. The goggles for supplying hydrogen according to claim 6, wherein
a coupling member for allowing inside and outside communication is arranged in both side parts of the goggle body, the goggle body and the temple member being coupled by coupling of a tip of each temple member with the coupling member,
the coupling member comprises, in order from an inner side,
an inner coupling part on the inner side of the goggle body,
a pedestal part to be mounted on both sides of the goggle body,
an outer coupling part which is coupled with the goggle body by sandwiching the pedestal part with the inner coupling part from both sides for coupling, the outer coupling part also being coupled with the temple member, and
an outer coupling part coupled with the outer coupling part, and fluidly connected with the discharging side end part of the tube member,
the inner coupling part, the pedestal part, the outer coupling part, and the outer coupling part fluidly connect the discharging side end part of the tube member and an inside of the goggles while being coupled to one another, and
a main body part of the temple member has a detaining part for detachably positioning the tube member.

8. The goggles for supplying hydrogen according to claim 6, wherein
the goggle body comprises an edge part which contacts a periphery of an eye of the user, and seals an inside and an outside of the goggle body, while providing a space between a front lens part and the periphery of the eye,
the hydrogen releasing part, which is adjustable to a position corresponding to each of the both lower eyelid parts, is arranged on a goggle body lower part side of the edge part, and
each hydrogen releasing part is fluidly connectable using a detachable short tube member, and either of the hydrogen releasing openings is fluidly connectable with a through hole, to which high-concentration hydrogen is supplied, using another detachable short tube member.

9. The method for promoting physiological action according to claim 2, wherein the air containing high-concentration hydrogen is released to the periphery of the eye, and percutaneous absorption of the hydrogen is caused to reduce brain fatigue.

10. The method for promoting physiological action according to claim 2, wherein the air containing high-concentration hydrogen is released to the periphery of the eye, and percutaneous absorption of the hydrogen is caused to enhance brain functions (improve age).

11. The method for promoting physiological action according to claim 2, wherein the air containing high-concentration hydrogen is released to the periphery of the eye, and percutaneous absorption of the hydrogen is caused to promote oxytocin secretion.

12. The goggles for supplying hydrogen according to claim 7, wherein
the goggle body comprises an edge part which contacts a periphery of an eye of the user, and seals an inside and an outside of the goggle body, while providing a space between a front lens part and the periphery of the eye,
the hydrogen releasing part, which is adjustable to a position corresponding to each of the both lower eyelid parts, is arranged on a goggle body lower part side of the edge part, and
each hydrogen releasing part is fluidly connectable using a detachable short tube member, and either of the hydrogen releasing openings is fluidly connectable with a through hole, to which high-concentration hydrogen is supplied, using another detachable short tube member.
